(19) United States
(12) Patent Application Publication
(10) Pub. No.: US 2025/0136610 A1
(43) Pub. Date: May 1, 2025

(54) CRYSTALLINE FORMS OF TRILACICLIB AND TRILACICLIB SALTS

(71) Applicant: TEVA PHARMACEUTICALS INTERNATIONAL GMBH, Jona (CH)

(72) Inventors: Anantha Rajmohan Muthusamy, Sivakasi (IN); Rahul Kumar Reddy Putikum, Mahabubnagar (IN); Prathap Rengaraj, Madurai (IN); Amit Singh, Greater Noida (IN); Rushikesh Vilasrao Kadu, Ahmedabad (IN); Satnam Singh, Greater Noida (IN)

(21) Appl. No.: 18/837,960

(22) PCT Filed: Feb. 28, 2023
(86) PCT No.: PCT/US2023/014031

§ 371 Date: Aug. 13, 2024

(30) Foreign Application Priority Data
Feb. 28, 2022 (IN) 202211010716
Oct. 7, 2022 (IN) 202211057476
Oct. 25, 2022 (IN) 202211060815

(51) Int. Cl. C07D 487/20; A61K 31/519

(52) U.S. Cl. CPC C07D 487/20; A61K 31/519

(57) ABSTRACT

The present disclosure encompasses solid state forms of Trilaciclib and Trilaciclib citrate salt, in embodiments poly-morphs of Trilaciclib and Trilaciclib citrate salt, processes for preparation thereof, pharmaceutical compositions and uses thereof.

### FIELD OF THE DISCLOSURE

The present disclosure encompasses solid state forms of Trilaciclib and Trilaciclib citrate salt, in embodiments polymorphs of Trilaciclib and Trilaciclib citrate salt, processes for preparation thereof, pharmaceutical compositions and uses thereof.

### BACKGROUND OF THE DISCLOSURE

Trilaciclib, has the following chemical structure:

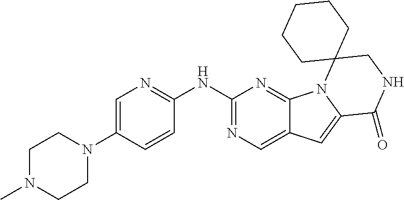 [SMILES: CN1CCN(c2ccc(Nc3ncc4cc5n(c4n3)C3(CCCCC3)CNC5=O)nc2)CC1]

Trilaciclib is a small molecule with potential antineoplastic and chemoprotective activities. In particular, it is investigated for myelopreservation (preserving bone marrow function) in patients with small cell lung cancer (SCLC) that receive chemotherapy. Trilaciclib is also under clinical investigation for myelopreservation (i.e., reducing bone marrow suppression) in patients with non-small cell lung cancer (NSCLC), colorectal cancer (particularly metastatic colorectal cancer), breast cancer (particularly metastatic triple-negative breast cancer), and bladder cancer (particularly advanced/metastatic bladder cancer), that receive chemotherapy.

The compound is described in U.S. Pat. No. 8,598,165.

International Publication No. WO2021/257587 discloses crystalline forms of Trilaciclib and Trilaciclib dihydrochloride salt. International Publication No. WO 2022076779 describes polymorphs of Trilaciclib; different salts of Trilaciclib and polymorphs thereof.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (“XRD” or “XRPD”) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Trilaciclib and of Trilaciclib salts.

### SUMMARY OF THE DISCLOSURE

The present disclosure provides crystalline polymorphs of Trilaciclib and Trilaciclib citrate salt, processes for preparation thereof, and pharmaceutical compositions thereof. These crystalline polymorphs can be used to prepare other forms of Trilaciclib or of Trilaciclib salts.

The present disclosure provides crystalline polymorphs of Trilaciclib and/or Trilaciclib citrate salt for use in the preparation of pharmaceutical compositions and/or formulations for use in medicine, in embodiments for the treatment of myelosuppression during chemotherapy, particularly for reducing chemotherapy-induced bone marrow suppression in patients with SCLC, NSCLC, colorectal cancer (particularly metastatic colorectal cancer), breast cancer (particularly metastatic triple-negative breast cancer), or bladder cancer (particularly advanced/metastatic bladder cancer); and particularly for reducing chemotherapy-induced bone marrow suppression in patients with SCLC.

The present disclosure provides crystalline polymorphs of Trilaciclib and/or Trilaciclib citrate salt for use in medicine, including as a chemoprotective agent (in particular; in patients suffering from SCLC, colorectal cancer, breast cancer (particularly metastatic triple-negative breast cancer), and bladder cancer (particularly advanced/metastatic bladder cancer), and particularly for reducing chemotherapy-induced bone marrow suppression in patients with SCLC, that receive chemotherapy), particularly for reducing chemotherapy-induced bone marrow suppression in patients with SCLC, NSCLC, colorectal cancer (particularly metastatic colorectal cancer), breast cancer (particularly metastatic triple negative breast cancer), or bladder cancer (particularly advanced/metastatic bladder cancer); and particularly for reducing chemotherapy-induced bone marrow suppression in patients with SCLC.

The present disclosure also encompasses the use of the solid state forms of Trilaciclib and/or Trilaciclib citrate salt of the present disclosure for the preparation of pharmaceutical compositions and/or formulations.

In another aspect, the present disclosure provides pharmaceutical compositions comprising any one or a combination of the polymorphs of Trilaciclib and/or Trilaciclib citrate salt according to the present disclosure.

The present disclosure includes processes for preparing the above mentioned pharmaceutical compositions. The processes include combining any one or a combination of the polymorphs of Trilacilib/Trilaciclib citrate salt with at least one pharmaceutically acceptable excipient.

The polymorphs of Trilaciclib and Trilaciclib citrate salt as defined herein and the pharmaceutical compositions or formulations of the polymorphs of Trilaciclib and/or Trilaciclib citrate salt may be used as medicaments, such as for the treatment of myelosuppression during chemotherapy.

The present disclosure also provides methods of treating myelosuppression during chemotherapy by administering a therapeutically effective amount of any one or a combination of the polymorphs of Trilaciclib/Trilaciclib citrate salt of the present disclosure, or at least one of the above pharmaceutical compositions, to a subject suffering from myelosuppression, or otherwise in need of the treatment.

The present disclosure also provides uses of polymorphs of Trilaciclib and/or Trilaciclib citrate salt of the present disclosure, or at least one of the above pharmaceutical compositions, for the manufacture of medicaments for treating, e.g., myelosuppression.

### BRIEF DESCRIPTION OF THE DRAWINGS

FIG. 1 shows a characteristic XRPD of Trilaciclib citrate salt-Form TCT2.

FIG. 2 shows a characteristic XRPD of amorphous Trilaciclib citrate salt.

FIG. 3 shows a characteristic XRPD of Trilaciclib citrate salt-Form TCT3.

FIG. 4 shows a characteristic XRPD of Trilaciclib-Form TT5.

FIG. 5 shows a characteristic XRPD of Trilaciclib-Form TT6.

FIG. 6 shows a characteristic XRPD of Trilaciclib-Form TT7.

FIG. 7 shows a characteristic solid state ¹³C NMR spectrum of Trilaciclib citrate salt-Form TCT3 (full screen).

FIG. 8: Solid state ¹³C NMR spectrum of Trilaciclib citrate salt-Form TCT3 (0-100 ppm).

FIG. 9: Solid state ¹³C NMR spectrum of Trilaciclib citrate salt-Form TCT3 (100-200 ppm).

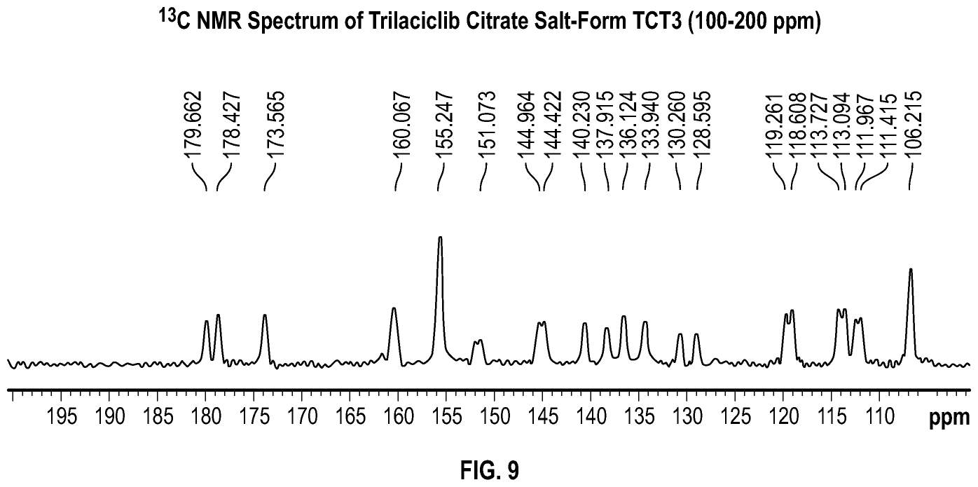

### DETAILED DESCRIPTION OF THE DISCLOSURE

The present disclosure encompasses solid state forms of Trilaciclib and Trilaciclib citrate salt, processes for preparation thereof, and pharmaceutical compositions thereof.

The solid state or polymorph forms of Trilaciclib or Trilaciclib citrate salt as described in any aspect or embodiment of the disclosure may be polymorphically pure or substantially free of any other forms.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. For example, any of the solid state forms of Trilaciclib citrate salt as described herein may be substantially free of any other solid state forms of Trilaciclib citrate salt. Similarly any of the solid state forms of Trilaciclib as described herein may be substantially free of any other solid state forms of Trilaciclib. Thus, a crystalline polymorph of Trilaciclib or of Trilaciclib salts described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Trilaciclib or of Trilaciclib salt. In some embodiments of the disclosure, the described crystalline polymorph of Trilaciclib or of Trilaciclib salt may contain from about 1% to about 20% (w/w), from about 5% to about 20% (w/w), or from about 5% to about 10% (w/w) of one or more other crystalline polymorph of Trilaciclib and/or of Trilaciclib salt.

Depending on which other crystalline polymorphs a comparison is made, the crystalline polymorphs of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Trilaciclib or salt of Trilaciclib referred to herein as being characterized by graphical data “as depicted in” or “as substantially depicted in” a Figure will thus be to include any crystal forms of Trilaciclib and of Trilaciclib salt characterized with the graphical data having such small variations, as are well known to the skilled person, in comparison with the Figure.

As used herein, and unless stated otherwise, the term “anhydrous” in relation to crystalline forms of Trilaciclib, relates to a crystalline form of Trilaciclib salt which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, unless stated otherwise, the XRPD measurements are taken using copper Kα radiation wavelength 1.5418 Å. XRPD peaks reported herein are measured using Cuk α radiation, λ=1.5418 Å, typically at a temperature of 25±3° C.

As used herein, unless stated otherwise, ¹³C NMR reported herein are measured at 11.7 T at a magic angle spinning frequency ωᵣ/2π=18 kHz, preferably at a temperature of at 300 K±3° C.

A thing, e.g., a reaction mixture, may be characterized herein as being at, or allowed to come to “room temperature” or “ambient temperature”, often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

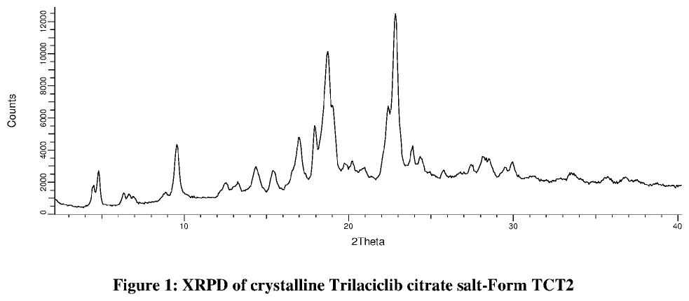

In one embodiment, the present disclosure includes a crystalline polymorph of Trilaciclib citrate salt; designated Form TCT2. The crystalline Form TCT2 may be characterized by data selected from one or more of the following: an X-ray powder diffraction pattern substantially as depicted in FIG. 1; an X-ray powder diffraction pattern having peaks at 4.8, 9.6, 14.3, 16.9 and 18.7 degrees 2-theta±0.2 degrees 2-theta; and combinations of these data.

Crystalline Form TCT2 of Trilaciclib citrate salt may be further characterized by an X-ray powder diffraction pattern having peaks at 4.8, 9.6, 14.3, 16.9 and 18.7 degrees 2-theta±0.2 degrees 2-theta, and also having any one, two, three or four additional peaks selected from 6.3, 12.5, 22.8 and 23.8 degrees 2-theta±0.2 degrees 2-theta.

In a further embodiment, crystalline Form TCT2 is characterized by an X-ray powder diffraction pattern having peaks at 4.8, 6.3, 9.6, 12.5, 14.3, 16.9, 18.7, 22.8 and 23.8 degrees 2-theta±0.2 degrees 2-theta.

Form TCT2 according to any aspect or embodiment of the present disclosure may be polymorphically pure.

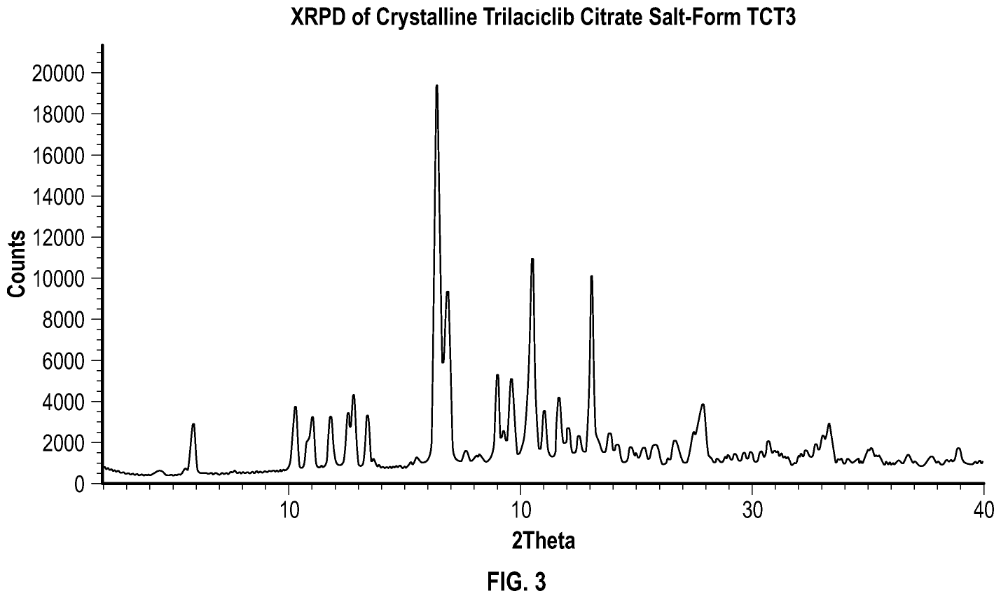

In another embodiment, the present disclosure includes a crystalline polymorph of Trilaciclib citrate salt; designated Form TCT3. The crystalline Form TCT3 may be characterized by data selected from one or more of the following: an X-ray powder diffraction pattern substantially as depicted in FIG. 3; an X-ray powder diffraction pattern having peaks at 10.3, 11.8, 16.4, 19.6 and 21.7 degrees 2-theta±0.2 degrees 2-theta; and combinations of these data.

Crystalline Form TCT3 of Trilaciclib citrate salt may be further characterized by an X-ray powder diffraction pattern having peaks at 10.3, 11.8, 16.4, 19.6 and 21.7 degrees 2-theta±0.2 degrees 2-theta, and also having any one, two, three or four additional peaks selected from 5.9, 13.4, 19.0 and 23.1 degrees 2-theta±0.2 degrees 2-theta.

In a further embodiment, crystalline Form TCT3 is characterized by an X-ray powder diffraction pattern having peaks at 5.9, 10.3, 11.8, 13.4, 16.4, 19.0, 19.6, 21.7, and 23.1 degrees 2-theta±0.2 degrees 2-theta.

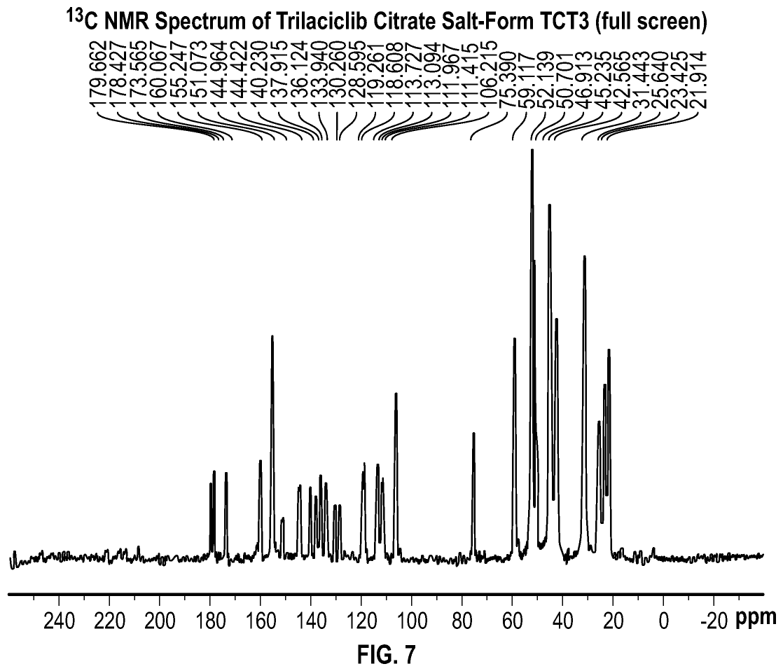

Crystalline Form TCT3 of Trilaciclib citrate salt as described in any aspect or embodiment of the disclosure may be alternatively or additionally characterized by a solid state ¹³C NMR spectrum with peaks at 31.4, 59.1, 75.4, 106.2, 155.2 and 160.1 ppm±0.2 ppm. Alternatively or additionally, crystalline Form TCT3 of Trilaciclib citrate salt may be characterized by a solid state ¹³C NMR spectrum having the following chemical shift absolute differences from a peak at 112.0 ppm±2 ppm of 80.6, 52.9, 36.6, 5.8, 43.2 and 48.1 ppm±0.1 ppm; optionally, Form TCT3 of Trilaciclib citrate salt may be characterized by a solid state ¹³C NMR spectrum substantially as depicted in any of FIG. 7, 8 or 9, preferably FIG. 7.

Crystalline Form TCT3 of Trilaciclib citrate salt may be a hemi citrate salt (i.e., the ratio Trilaciclib: citric acid is 2:1; accordingly). Most preferably, Form TCT3 as described in any aspect or embodiment of the disclosure is a Trilaciclib hemicitrate salt.

Form TCT3 according to any aspect or embodiment of the present disclosure is anhydrous.

Form TCT3 according to any aspect or embodiment of the present disclosure may be polymorphically pure.

According to any aspect or embodiment of the disclosure, crystalline Form TCT3 of Trilaciclib hemi-citrate salt may be provided in a particular morphology. In particular, the crystalline Form TCT3 may comprise particles having lath-shaped morphology. Crystalline Form TCT3 having the morphology as described herein provides several benefits for pharmaceutical drugs, particularly for improving their solubility, stability, and bioavailability.

Crystalline Form TCT3 of Trilaciclib hemi-citrate salt is stable under all tested stress conditions (e.g., under strong grinding, pressure of 2 tons, high humidity (up to 100% RH for 7 days) and at high temperature (up to 100° C.).

The above crystalline polymorphs of Trilaciclib citrate salt can be used to prepare other crystalline polymorphs of Trilaciclib, other Trilaciclib salts and solid state forms thereof.

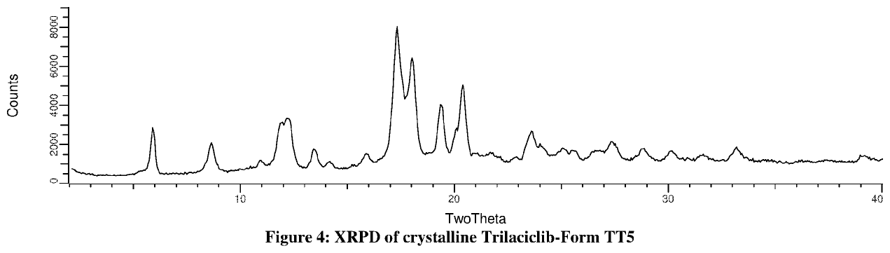

In a further embodiment, the present invention discloses a crystalline polymorph of Trilaciclib; designated Form TT5. The crystalline From TT5 of Trilaciclib may be characterized by data selected from one or more of the following: an X-ray powder diffraction pattern substantially as depicted in FIG. 4; an X-ray powder diffraction pattern having peaks at 5.9, 12.2, 18.0, 19.4 and 20.4 degrees 2-theta±0.2 degrees 2-theta; and combinations of these data.

Crystalline Form TT5 of Trilaciclib may be further characterized by an X-ray powder diffraction pattern having peaks at 5.9, 12.2, 18.0, 19.4 and 20.4 degrees 2-theta±0.2 degrees 2-theta, and also having any one, two or three additional peaks selected from 8.7, 13.5 and 23.6 degrees 2-theta±0.2 degrees 2-theta.

In a further embodiment crystalline Form TT5 of Trilaciclib may be characterized by an X-ray powder diffraction pattern having peaks at 5.9, 8.7, 12.2, 13.5, 18.0, 19.4, 20.4 and 23.6 degrees 2-theta±0.2 degrees 2-theta.

Form TT5 of Trilaciclib may be anhydrous.

Form TT5 according to any aspect or embodiment of the present disclosure may be polymorphically pure.

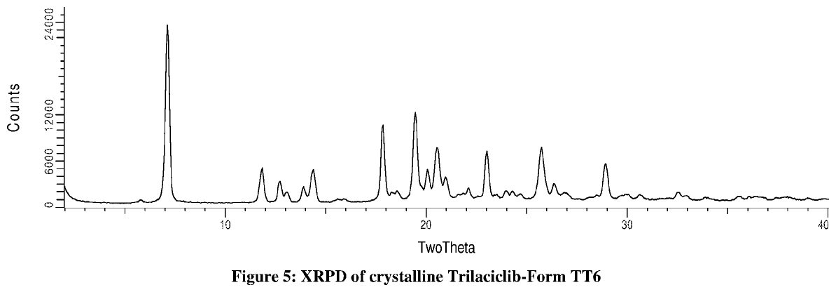

In another embodiment, the present invention discloses a crystalline polymorph of Trilaciclib; designated Form TT6. The crystalline From TT6 of Trilaciclib may be characterized by data selected from one or more of the following: an X-ray powder diffraction pattern substantially as depicted in FIG. 5; an X-ray powder diffraction pattern having peaks at 7.3, 14.5, 19.6, 20.7 and 25.9 degrees 2-theta±0.2 degrees 2-theta; and combinations of these data.

Crystalline Form TT6 of Trilaciclib may be further characterized by an X-ray powder diffraction pattern having peaks at 7.3, 14.5, 19.6, 20.7 and 25.9 degrees 2-theta±0.2 degrees 2-theta, and also having any one, two, three, or four additional peaks selected from 12.0, 12.9, 23.2 and 29.1 degrees 2-theta±0.2 degrees 2-theta.

In a further embodiment Crystalline Form TT6 of Trilaciclib may be further characterized by an X-ray powder diffraction pattern having peaks at 7.3, 12.0, 12.9, 14.5, 19.6, 20.7, 23.2, 25.9 and 29.1 degrees 2-theta±0.2 degrees 2-theta.

Form TT6 of Trilaciclib may be hydrate.

Form TT6 according to any aspect or embodiment of the present disclosure may be polymorphically pure.

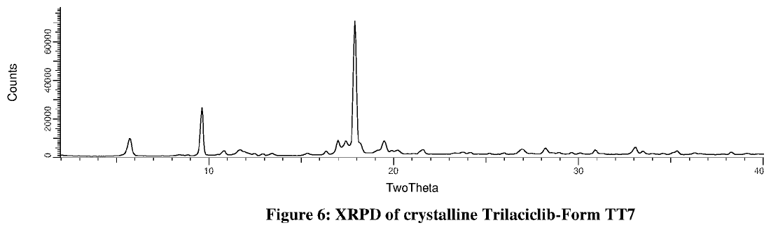

In a further embodiment, the present invention discloses a crystalline polymorph of Trilaciclib; designated Form TT7. The crystalline From TT7 of Trilaciclib may be characterized by data selected from one or more of the following: an X-ray powder diffraction pattern substantially as depicted in FIG. 6; an X-ray powder diffraction pattern having peaks at 5.8, 9.7, 18.0, 19.6 and 27.1 degrees 2-theta±0.2 degrees 2-theta; and combinations of these data.

Crystalline Form TT7 of Trilaciclib may be further characterized by an X-ray powder diffraction pattern having peaks at 5.8, 9.7, 18.0, 19.6 and 27.1 degrees 2-theta±0.2 degrees 2-theta, and also having any one, two, three or four additional peaks selected from 10.9, 21.7, 28.3 and 33.2 degrees 2-theta±0.2 degrees 2-theta.

In another embodiment Crystalline Form TT7 of Trilaciclib may be characterized by an X-ray powder diffraction pattern having peaks at 5.8, 9.7, 10.9, 18.0, 19.6, 21.7, 27.1, 28.3 and 33.2 degrees 2-theta±0.2 degrees 2-theta.

Form TT7 of Trilaciclib may be hydrate.

Form TT7 according to any aspect or embodiment of the present disclosure may be polymorphically pure.

The present disclosure provides crystalline polymorphs of Trilaciclib or Trilaciclib citrate salt for use in the preparation of pharmaceutical compositions including Trilaciclib or Trilaciclib salts and/or crystalline polymorphs thereof.

The present disclosure also encompasses the use of the solid state forms of Trilaciclib and/or Trilaciclib citrate salt according to any aspect or embodiment of the present disclosure for the preparation of pharmaceutical compositions and/or formulations. The pharmaceutical compositions and/or formulations may contain a Trilaciclib salt other than, or in addition to, the citrate salt, particularly Trilaciclib hydrochloride, Trilaciclib dihydrochloride, Trilaciclib sulfate, and Trilaciclib hydrobromide, and more particularly dihydrochloride.

The present disclosure further encompasses pharmaceutical compositions and/or formulations comprising a pharmaceutically acceptable salt of Trilaciclib, particularly Trilaciclib citrate, Trilaciclib Trilaciclib hemicitrate, hydrochloride, Trilaciclib dihydrochloride, Trilaciclib sulfate, and Trilaciclib hydrobromide, and more particularly dihydrochloride, which is prepared using a crystalline polymorph of Trilaciclib or Trilaciclib citrate salt of any aspect or embodiment of the present disclosure.

The present disclosure also encompasses the use of crystalline polymorphs of Trilaciclib or Trilaciclib citrate salt of the present disclosure for the preparation of pharmaceutical compositions of Trilaciclib or pharmaceutically acceptable salt of Trilaciclib. The process for the preparation of the pharmaceutical compositions may be as described herein below. The pharmaceutical composition or formulation can comprise a pharmaceutically acceptable salt of Trilaciclib, preferably Trilaciclib citrate, Trilaciclib hemicitrate, Trilaciclib hydrochloride, Trilaciclib dihydrochloride, Trilaciclib sulfate, or Trilaciclib hydrobromide; particularly Trilaciclib hemicitrate, Trilaciclib citrate or Trilaciclib dihydrochloride; and more particularly Trilaciclib dihydrochloride. The compositions or formulations can be prepared by converting the Trilaciclib or Trilaciclib citrate salt of any aspect or embodiment of the present disclosure to the pharmaceutically acceptable salt of Trilaciclib, either prior to the formulation process, or in situ during the formulation process (for example by salt formation). In particular, the present disclosure provides the use of a crystalline polymorph or Trilaciclib or Trilaciclib citrate according to any aspect or embodiment of the disclosure for preparing a pharmaceutical composition or formulation, wherein the composition or formulation comprises a pharmaceutically acceptable salt of Trilaciclib, preferably Trilaciclib citrate, Trilaciclib hemicitrate, Trilaciclib hydrochloride, Trilaciclib dihydrochloride, Trilaciclib sulfate, or Trilaciclib hydrobromide; particularly Trilaciclib hemicitrate, Trilaciclib citrate or Trilaciclib dihydrochloride; and more particularly Trilaciclib dihydrochloride

The present disclosure includes processes for preparing the above-mentioned pharmaceutical compositions. The processes include combining polymorph of Trilaciclib or Trilaciclib citrate salt of the present disclosure with at least one pharmaceutically acceptable excipient. Suitable pharmaceutically acceptable excipients may comprise bulking agents and buffering agents. Particularly, the pharmaceutical composition is a lyophilized powder. In particular, the process may comprise combining Trilaciclib hemicitrate salt, preferably Form TCT3 as defined in any aspect or embodiment of the present disclosure, with: at least one bulking agent, at least one buffering agent, a lyophilization solvent, and one or more pH adjustment agents (preferably an acid and a base), to form a solution; and lyophilizing the solution. According to any aspect or embodiment of this process, the bulking agent may be selected from the group consisting of: glycine, arginine, histidine, mannitol, sorbitol, lactose, sucrose, trehalose, glucose, raffinose, dextran, and polyethylene glycol; preferably glycine or mannitol; and more preferably mannitol. According to any aspect or embodiment of this process, the buffering agent can be selected from the group consisting of: citric acid, sodium citrate, potassium citrate, tartaric acid, sodium phosphate, Tris base, Tris HCl, Tris acetate, sodium acetate, potassium acetate, or arginine; preferably citric acid, sodium citrate, potassium citrate, tartaric acid or sodium phosphate; and more preferably citric acid or sodium citrate; especially citric acid. Any suitable pH adjustment agent may be used to adjust the pH of the solution. Preferably, the pH adjustment agent comprises a mineral acid, preferably selected from the group consisting of: hydrochloric acid, sulfuric acid, nitric acid, phosphoric acid; preferably hydrochloric acid or sulfuric acid; and most preferably hydrochloric acid. The pH adjustment agent comprises a base, preferably selected from sodium hydroxide or meglumine. Particularly, according to any aspect or embodiment of this method, the pH adjustment agent comprises hydrochloric acid and sodium hydroxide. Water is a particularly suitable lyophilization solvent for this process.

In a preferred embodiment, the disclosure provides a process for preparing a pharmaceutical composition or formulation comprising: (a) combining crystalline Trilaciclib hemicitrate salt as described in any aspect or embodiment of the disclosure, with at least one bulking agent and at least one buffering agent, in the lyophilization solvent; (b) adjusting the pH of the mixture to: about 1.6 to about 3.2, about 1.8 to about 3.0, about 1.9 to about 2.8, about 2.0 to about 2.5; about 2.1 to about 2.4; or about 2.2 to about 2.3; or about 2.25; (c) adjusting the pH of the mixture to: about 3.4 to about 5.8, about 3.6 to about 5.5, about 3.8 to about 5.0; about 4.0 to about 4.8; about 4.1 to about 4.6, about 4.2 to about 4.5, about 4.3 to about 4.4, or about 4.3; and (d) lyophilizing. Preferably, the pH adjustment agent in step (b) is hydrochloric acid, more preferably concentrated hydrochloric acid. Preferably, the pH adjustment agent in step (c) is sodium hydroxide; and the lyophilization solvent is water.

Preferably, the bulking agent is mannitol; the buffering agent is citric acid; the pH adjustment agents are hydrochloric acid, preferably concentrated hydrochloric acid, and sodium hydroxide; and the lyophilization solvent is water.

The present disclosure further provides a pharmaceutical composition or formulation, which is obtainable by a process according to any aspect or embodiment as disclosed herein.

The polymorphs of Trilaciclib or Trilaciclib citrate salt and the pharmaceutical compositions and/or formulations of Trilaciclib and of Trilaciclib salts of the present disclosure, can be used as medicaments. Particularly, according to any aspect or embodiment of the disclosure, the polymorphs of Trilaciclib or Trilaciclib citrate salt and the pharmaceutical compositions and/or formulations of Trilaciclib and of Trilaciclib salts of the present disclosure, can be used for the treatment of myelosuppression during chemotherapy, particularly for reducing chemotherapy-induced bone marrow suppression in patients with SCLC, NSCLC, colorectal cancer (particularly metastatic colorectal cancer), breast cancer (particularly metastatic triple-negative breast cancer), or bladder cancer (particularly advanced/metastatic bladder cancer); and particularly for reducing chemotherapy-induced bone marrow suppression in patients with SCLC.

The present disclosure also provides methods of treating myelosuppression by administering a therapeutically effective amount of any one or a combination of the crystalline polymorphs of Trilaciclib and/or Trilaciclib salts of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”) Method

X-ray diffraction was performed on X-Ray powder diffractometer: Bruker D8 Advance; CuKα radiation (λ=1.5418 Å); Lynx eye detector; laboratory temperature 22-25° C.; PMMA specimen holder ring with silicon low background. Prior to analysis, the samples were gently ground by means of mortar and pestle in order to obtain a fine powder. The ground sample was adjusted into a cavity of the sample holder and the surface of the sample was smoothed by means of a cover glass.

### Measurement Parameters:

Scan range: 2-40 degrees 2-theta;
Scan mode: continuous;
Step size: 0.05 degrees;
Time per step: 0.5 s;
Sample spin: 30 rpm;
Sample holder: PMMA specimen holder ring with silicon low background.
All X-Ray Powder Diffraction peak values are calibrated with regard to standard silicon spiking in the sample.
   ssNMR Method:

Solid-state NMR spectra were measured at 11.7 T using a Bruker Avance III HD 500 US/WB NMR spectrometer (Karlsruhe, Germany, 2013) with 3.2 mm probehead. The ¹³C CP/MAS NMR spectra employing cross-polarization were acquired using the standard pulse scheme at spinning frequency of 18 kHz and a room temperature (300 K). The recycle delay was 8 s and the cross-polarization contact time was 2 ms. The ¹³C scale was referenced to α-glycine (176.03 ppm for ¹³C). Frictional heating of the spinning samples was offset by active cooling, and the temperature calibration was performed with Pb(NO₃)₂. The NMR spectrometer was completely calibrated and all experimental parameters were carefully optimized prior the investigation. Magic angle was set using KBr during standard optimization procedure and homogeneity of magnetic field was optimized using adamantane sample (resulting line-width at half-height Δν1/2 was less than 3.5 Hz at 250 ms of acquisition time).

### Optical Microscope:

Optical microscopy was performed using a Leica microscope. Sample was taken in a glass vial, silicon oil was added and sample was vortexed for 10 seconds. A drop of homogenous sample solution place on glass slide and covered with coverslip.

### Examples

### Preparation of Starting Materials

Trilaciclib can be prepared according to methods known from the literature, for example U.S. Pat. No. 8,598,186.

Form TT1 of Trilaciclib and Form THCl1 of Trilaciclib diHCl can be prepared according to International Publication No. WO20220076779.

### Example 1: Preparation of Trilaciclib Citrate Salt Form TCT2

Trilaciclib (2.5 grams) was dissolved in a mixture of Dichloromethane: Methanol (80:20 v/v; 500 mL) at 60° C. The reaction mixture was cooled down to 25° C. and then citric acid (1.07 grams, 1 mole eq.) was added and the mixture was stirred at 25° C. for about 20 hours. The mixture was cooled to 5° C., acetone (500 mL) was added and stirred for about 1 hour at 5° C. The reaction mass was allowed to warm to room temperature, filtered and dried under vacuum for 30-45 minutes at 25° C. The obtained solid was dried in a vacuum tray dryer at 60° C. for about 6 hours. The obtained solid was analyzed by XRPD and designated as Form TCT2 of Trilaciclib citrate salt; as shown in FIG. 1.

### Example 2: Preparation of Amorphous Trilaciclib Citrate Salt

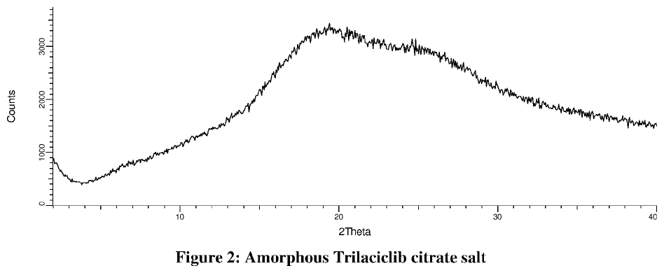

Trilaciclib citrate salt (Form TCT2, 0.5 grams) was dissolved in water (150 mL). The solution was frozen in liquid nitrogen and the frozen solid mass was subjected to lyophilization (condensation temp −107° C. and vacuum up to 50 mTorr). After 57 hours solid sample was isolated at 25° C. The obtained solid was analyzed by XRPD, amorphous Trilaciclib citrate salt; as shown in FIG. 2.

### Example 3: Preparation of Trilaciclib Citrate Salt Form TCT3

A mixture of Trilaciclib (Form TT1, 0.5 grams), citric acid monohydrate (0.124 grams) and water (2.5 ml) was heated up to 55-65° C. and stirred for about 30 minutes. Acetone (10 ml) was then added, the reaction mixture was stirred for about 1 hour at 55-65° C., cool down to 20-25° C. and stirred for additional 2 hours. The reaction mass was filtered, washed with acetone (1 ml) and dried under vacuum at 50-60° C. The obtained solid (0.5 grams) was analyzed by XRPD and designated as Trilaciclib citrate salt Form TCT3; as shown in Figure

### Example 4: Preparation of Trilaciclib-Form TT5

Trilaciclib dihydrochloride salt (THC11, 0.05 grams) was dissolved in water (2 ml) at 50-55° C. to obtain a clear solution. The clear solution was then cooled to about 10° C. (during 10-15 minutes), dropwise was added triethylamine (0.1 ml) and solution was stirred about 1 hour at 10° C. The obtained solid was filtered at room temp and dried under vacuum (about 1 hour). The dried sample was analyzed by XRPD and designated as Form TT5 of Trilaciclib, as shown in FIG. 4.

### Example 5: Preparation of Trilaciclib-Form TT6

Trilaciclib dihydrochloride salt (THC11, 2.0 grams) was dissolved in water (80 ml) at 50-55° C. to obtain a clear solution. The clear solution was then cooled to about 10° C. (during 30-45 minutes), dropwise was added triethylamine (4 ml) and solution was stirred about 24 hours at 10° C. The obtained solid was filtered at room temperature and dried under vacuum for about 1 hour. The dried sample was analyzed by XRPD and designated as Form TT6 of Trilaciclib; as shown in FIG. 5.

### Example 6: Preparation of Trilaciclib-Form TT7

Trilaciclib dihydrochloride salt (THC11, 0.05 grams) was dissolved in water (2 ml) at 50-55° C. to obtain a clear solution. The clear solution was cooled to 10° C. (during 10-15 minutes), dropwise was added IN NaOH solution (0.1 ml) and solution was stirred for about 1 hour at 10° C. The obtained solid was filtered at room temperature and dried under vacuum for about 1 hour. The dried sample was analyzed by XRPD and designated as Form TT7 of Trilaciclib; as shown in FIG. 6.

### Example 7: Stability Experiments

### Storage Stability at Different Relative Humidities

Samples of Trilaciclib citrate Form TCT3 were subjected to conditions of different relative humidities at ambient temperature. XRPD analysis was performed on the samples after 7 days. The results are shown in Table 1 below:

TABLE 1

| Trilaciclib | Relative humidity | | | | |
|---|---|---|---|---|---|
| citrate Form TCT3 | 20% | 40% | 60% | 80% | 100% |
| XRPD | No | No | No | No | No |
| analysis | polymorphic | polymorphic | polymorphic | polymorphic | polymorphic |
| results | conversion | conversion | conversion | conversion | conversion |

These results demonstrate that Trilaciclib citrate Form TCT3 is especially stable to high and low relative humidity conditions and is particularly suitable for use in pharmaceutical dosage forms.

Samples of Trilaciclib citrate Form TCT3 were subjected to conditions of different relative humidities at different temperatures. XRPD analysis was performed on the samples after 2 months. The results are shown in Table 2 below:

TABLE 2

Conditions (2 months)

| Trilaciclib | 25° C., | 40° C., |
|---|---|---|
| citrate Form TCT3 | 60% RH | 75% RH |
| XRPD | No | No |
| analysis | polymorphic | polymorphic |
| results | conversion | conversion |

The results demonstrate that Trilaciclib citrate Form TCT3 shows no polymorphic conversion after exposure to high and low relative humidity at different temperatures for at least 2 months, indicating that this crystalline form has good storage stability. Form TCT3 of Trilaciclib citrate was also found to be stable for at least 2 months at 2-8° C. Moreover, Form TCT3 is stable for at least 3 months at room temperature and humidity.

### Grinding Experiments

Samples of Trilaciclib citrate Form TCT3 were subjected to strong grinding, and to solvent drop grinding in isopropanol, ethanol and water. Grinding was carried out on the samples alone, or in the presence of ethanol or water. In these experiments, about 20 mg of the sample is placed in a mortar and ground with a pestle for 2 minutes. The solvent, when used, as added to the crystalline material before grinding, in a volume of 10 microliters. XRPD analysis performed on each of the samples after the grinding experiment, confirmed no change in the starting material (Table 3):

TABLE 3
Trilaciclib citrate Form TCT3

| Condition | XRPD analysis results |
|---|---|
| Strong grinding | No polymorphic conversion |
| Solvent-drop grinding (ethanol) | No polymorphic conversion |
| Solvent-drop grinding (isopropanol) | No polymorphic conversion |
| Solvent-drop grinding (water) | No polymorphic conversion |

The results demonstrate that Trilaciclib citrate Form TCT3 is resistant to polymorphic changes and is highly suitable for preparing pharmaceutical formulations.

### Thermal Stability

A sample of Trilaciclib citrate Form TCT3 was subjected to heating up to 100° C. for 30 minutes. XRPD analysis of the sample confirmed no change in the starting material (Table 4):

TABLE 4
Trilaciclib citrate Form TCT3

| Condition | Result |
|---|---|
| Heating 100° C., 30 minutes | No polymorphic conversion |

### Stability to Compression

Samples of Trilaciclib citrate Form TCT3 were subjected to a pressure of 2 tons (Atlas® Autopress hydraulic press, set to 2 tons). XRPD analysis was performed on the samples after 1-5 minutes. The results are shown in Table 5 below:

TABLE 5

| XRPD analysis results | Pressure (2 tons) |
|---|---|
| Trilaciclib citrate | No polymorphic |
| Form TCT3 | conversion |

1. Crystalline Trilaciclib hemicitrate salt.

2. A crystalline form of Trilaciclib hemicitrate salt designated Form TCT3, which is characterized by data selected from one or more of the following:
(a) an X-ray powder diffraction pattern substantially as depicted in FIG. 3;
(b) an X-ray powder diffraction pattern having peaks at 10.3, 11.8, 16.4, 19.6 and 21.7 degrees 2-theta±0.2 degrees 2-theta;
(c) a solid state ¹³C NMR spectrum with peaks at 31.4, 59.1, 75.4, 106.2, 155.2 and 160.1 ppm±0.2 ppm;
(d) a solid state ¹³C NMR spectrum having the following chemical shift absolute differences from a peak at 112.0 ppm±2 ppm of 80.6, 52.9, 36.6, 5.8, 43.2 and 48.1 ppm±0.1 ppm;
(e) a solid state ¹³C NMR spectrum substantially as depicted in any of FIG. 7, 8 or 9;
and combinations of these data.

3. The crystalline form of Trilaciclib hemicitrate salt according to claim 2, which is characterized by data selected from one or more of the following:
(a) an X-ray powder diffraction pattern substantially as depicted in FIG. 3; or
(b) an X-ray powder diffraction pattern having peaks at 10.3, 11.8, 16.4, 19.6 and 21.7 degrees 2-theta±0.2 degrees 2-theta.

4. The crystalline form of Trilaciclib hemicitrate salt according claim 2, which is further characterized by an X-ray powder diffraction pattern having any one, two, three or four additional peaks selected from 5.9, 13.4, 19.0 and 23.1 degrees 2-theta±0.2 degrees 2-theta.

5. The crystalline form of Trilaciclib hemicitrate salt according to claim 3, which is further characterized by a solid state ¹³C NMR spectrum with peaks at 31.4, 59.1, 75.4, 106.2, 155.2 and 160.1 ppm±0.2 ppm.

6. The crystalline form of Trilaciclib hemicitrate salt according to claim 2, which is further characterized by or a solid state ¹³C NMR spectrum having the following chemical shift absolute differences from a peak at 112.0 ppm±2 ppm of 80.6, 52.9, 36.6, 5.8, 43.2 and 48.1 ppm±0.1 ppm.

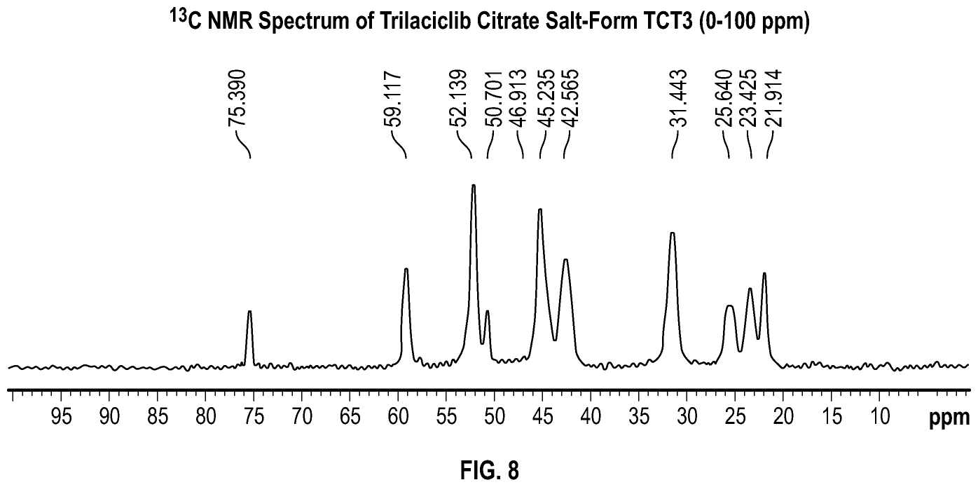

7. The crystalline form of Trilaciclib hemicitrate salt according to claim 2, which is further characterized by or a solid state ¹³C NMR spectrum substantially as depicted in any of FIGS. 7, 8, and/or 9.

8. The crystalline form of Trilaciclib hemicitrate salt according to claim 2, which is characterized by an XRPD pattern having peaks at 5.9, 10.3, 11.8, 13.4, 16.4, 19.0, 19.6, 21.7, and 23.1 degrees 2-theta±0.2 degrees 2-theta.

9. The crystalline form of Trilaciclib hemicitrate salt according to claim 2, which is substantially free of any other solid state forms of Trilaciclib and/or Trilaciclib salt.

10. The crystalline form of Trilaciclib hemicitrate salt according to claim 2, containing about 20% (w/w) or less, about 10% (w/w) or less, about 5% (w/w) or less, about 2% (w/w) or less, about 1% (w/w) or less, or about 0% (w/w) of any other crystalline forms of Trilaciclib and/or Trilaciclib salt.

11. The crystalline form of Trilaciclib hemicitrate salt according to claim 2, containing about 20% (w/w) or less, about 10% (w/w) or less, about 5% (w/w) or less, about 2% (w/w) or less, about 1% (w/w) or less, or about 0% (w/w) of amorphous forms of Trilaciclib and/or Trilaciclib salt.

12. A method for preparing other crystalline forms of Trilaciclib, salts of Trilaciclib or crystalline forms thereof using the crystalline Trilaciclib hemicitrate salt of claim 1.

13. A pharmaceutical composition comprising crystalline Trilaciclib hemicitrate salt according to claim 1, and at least one pharmaceutically acceptable excipient.

14. A process for preparation of a pharmaceutical composition or formulation using the crystalline Trilaciclib hemicitrate salt of claim 1.

15. The process according to claim 14, wherein the composition or formulation comprises a pharmaceutically acceptable salt of Trilaciclib, including at least one of Trilaciclib citrate, Trilaciclib hemicitrate, Trilaciclib hydrochloride, Trilaciclib dihydrochloride, Trilaciclib sulfate, or Trilaciclib hydrobromide.

16. A method of treating a condition, comprising administering a therapeutically effective amount of crystalline Trilaciclib hemicitrate salt according to claim 1 as a medicament to a subject in need of the treatment.

17. A method of treating a condition, comprising administering a therapeutically effective amount of crystalline Trilaciclib hemicitrate salt according to claim 1 as a chemo protective agent to reduce chemotherapy-induced bone marrow suppression in patients with small cell lung cancer (SCLC), non-small cell lung cancer (NSCLC), colorectal cancer, breast cancer, or bladder cancer.

18. A method of treating chemotherapy-induced bone marrow suppression comprising administering a therapeutically effective amount of crystalline Trilaciclib dihydrochloride salt according to claim 1, to a subject in need of the treatment.
